(19) United States
(12) Patent Application Publication
(10) Pub. No.: US 2005/0165475 A1
(43) Pub. Date: Jul. 28, 2005

(54) Hybrid grafts including biodegradable polymer supporting layer and manufacturing process thereof

(76) Inventor: In-Sup Noh, Seoul (KR)

Correspondence Address: JONES DAY, 222 EAST 41ST ST, NEW YORK, NY, 10017, US

(21) Appl. No.: 10/509,314

(22) PCT Filed: Mar. 27, 2003
(86) PCT No.: PCT/KR03/00611

(30) Foreign Application Priority Data
Mar. 27, 2002 (KR) 10-2002-0016866

(51) Int. Cl. A61F 2/06; B05D 3/00

(52) U.S. Cl. 623/1.38; 623/1.42; 427/2.25

(57) ABSTRACT

The present invention relates to a hybrid artificial blood vessel including a biodegradable polymer supporting layer and the manufacturing process thereof. There is provided a hybrid artificial blood vessel and a manufacturing process thereof, constructing a biodegradable supporting layer on the inner and outer surface of the hybrid artificial blood vessel, thereby increasing biocompatibility and enhancing circulation of blood vessel.

### FIELD OF THE INVENTION

The present invention relates to a hybrid artificial blood vessel comprising a biodegradable polymer-supporting layer, and more particularly to a hybrid artificial blood vessel and a manufacturing process for the hybrid artificial blood vessel, which comprises a drug and a biodegradable supporting layer on the inner and outer surfaces of the vessel to improve the biocompatiblility and patency of the artificial blood vessel.

### DESCRIPTION OF THE PRIOR ART

As generally known in the art, artificial blood vessels are artificial-organs used for the purpose of repairing the blocked circulation in vivo. Since artificial blood vessels have the characteristic of being permanently implanted in the body, they are required to have high safety and be composed of materials having good biocompatibility and histocompatibility.

Artificial blood vessels for medical use currently available on the market include those made of PET or drawn polytetrafluoroethylene film (hereinafter, referred to ‘ePTFE’) and those originating in living tissue. Particularly, the ePTFE is a polymer material used for artificial blood vessels having a microporous structure of a micron unit. The ePTFE having a large diameter having an inner diameter of at least 5 mm is commercially available for transplantation use in patients, but the ePTFE having a small diameter has a difficulty for use as an artificial blood vessel because of low patency after transplant.

Meanwhile, the existing ePTFE processing methods include a plasma treatment and a method inducing cell adhesion on ePTFE surface by coating cell adhesion-inducing proteins on the surface, but the methods have been limited for use in transplantation in patients for a long time because cell cultures are detached from the surface of artificial blood vessels if transplanted into blood vessels in the body.

Therefore, there is a need for an ePTFE artificial blood vessel for tissue engineering use to improve the biocompatibility and patency of the ePTFE artificial blood vessel.

### SUMMARY OF THE INVENTION

Accordingly, the present invention has been made to solve the above-mentioned problems occurring in the prior art, and an object of the present invention is to provide a hybrid artificial blood vessel, which includes a biodegradable supporting layer.

It is another object of the present invention to provide a method for manufacturing a hybrid artificial blood vessel, which includes a biodegradable supporting layer.

In order to accomplish the first object, there is provided a hybrid artificial blood vessel comprising biodegradable polymer-supporting layers on at least one of inner and outer surfaces of non-degradable artificial blood vessels.

In the hybrid artificial blood vessel, the biodegradable polymer is preferably at least one polymer selected from the group consisting of polyglycolide, polylactid, PLGA [Poly(Lactic-co-Glycolic Acid)], chitosan, gelatin, alginic acid and collagen.

In the hybrid artificial blood vessel, the non-degradable artificial blood vessels preferably comprise polyurethane derivatives, DacronR or drawn polytetrafluoroethylene.

According to an embodiment of the present invention, the hybrid artificial blood vessel further contains a drug which is preferably stored in at least one region selected from the microporous space of the non-degradable artificial blood vessel layer, biodegradable polymer-supporting layers, and the interfaces of the artificial blood vessel layers and the supporting layers.

In this embodiment of the present invention, the drug preferably comprises at least one selected from vascular endothelial growth factor, fibroblast growth factor, nerve growth factor, platelet-derived growth factor, heparin, thrombin, laminin, fibronectin and collagen.

In another embodiment of the present invention, the biodegradable polymer-supporting layer is preferably porous.

In yet another embodiment of the present invention, the biodegradable polymer-supporting layers are preferably formed on the artificial blood vessel layer by repetitive coating.

In yet another embodiment of the present invention, the surfaces of the non-degradable artificial blood vessel layer are preferably modified physically or chemically.

In order to accomplish the second object, there is provided a manufacturing process of a hybrid artificial blood vessel comprising the steps: dissolving biodegradable polymer in organic solvent to prepare biodegradable polymer solution A; adding porogen to the polymer solution A; dissolving the same or different biodegradable polymer with the above biodegradable polymer in organic solvent to prepare biodegradable polymer solution B; incorporating the biodegradable polymer solution B into the micropores of an artificial blood vessel layer; inserting tubes to the inside and outside of the artificial blood vessel layer; filling the biodegradable polymer solution A in the space between the artificial blood vessel layer and the tubes; drying the artificial blood vessel layer filled with the biodegradable polymer solution A to remove the organic solvent; and incubating the artificial blood vessel layer filled with the biodegradable polymer solution A in a water bath to remove the porogen.

### BRIEF DESCRIPTION OF THE DRAWINGS

The above and other objects, features and advantages of the present invention will be more apparent from the following detailed description taken in conjunction with the accompanying drawings, in which:

FIG. 1 is a scheme for the manufacturing process of a hybrid artificial blood vessel comprising biodegradable polymer-supporting layers according to one embodiment of the present invention;

FIG. 2 is a sectional drawing of a hybrid artificial blood vessel comprising biodegradable polymer-supporting layers according to one embodiment of the present invention;

FIG. 3 is a scanning electron micrograph showing a section of a hybrid artificial blood vessel according to one embodiment of the present invention;

FIG. 4 is a X-ray photoelectron micrograph showing the exterior surface of a hybrid artificial blood vessel comprising a biodegradable polymer-supporting layer according to one embodiment of the present invention;

FIG. 5 is a scanning electron micrograph showing a section of an artificial blood vessel layer before adding a biodegradable polymer-supporting layer according to one embodiment of the present invention; and

FIG. 6 is an X-ray photoelectron micrograph showing the exterior surface of an artificial blood vessel layer before adding a biodegradable polymer-supporting layer according to one embodiment of the present invention.

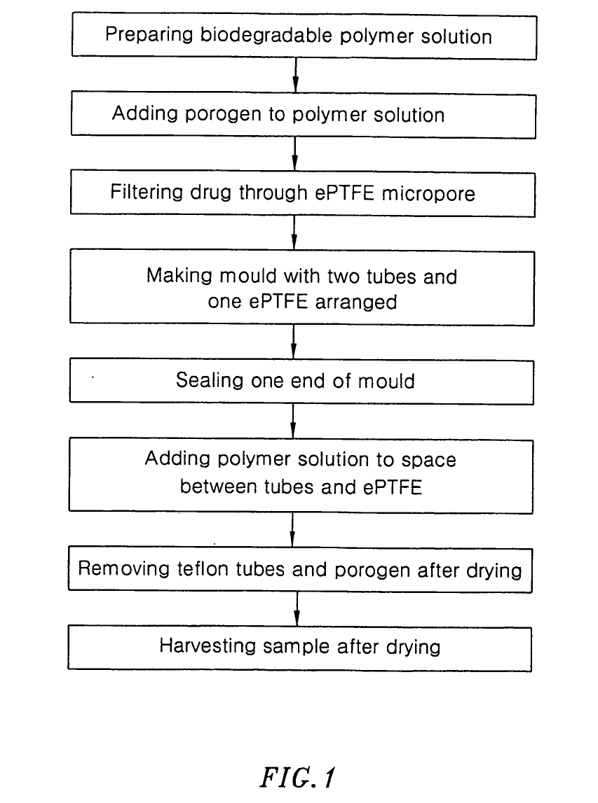

### DETAILED DESCRIPTION OF THE INVENTION

The present invention is directed to an artificial blood vessel to improve patency of the blood vessel, particularly regarding an artificial blood vessel of a hybrid type comprising biodegradable polymer-supporting layers. More specifically, the present invention is directed to a hybrid artificial blood vessel capable of forming vascular tissues on a non-degradable artificial blood vessel layer by reinforcing the non-degradable artificial blood vessel layer with a drug and with biodegradable polymer-supporting layers and then culturing blood vessel cells seeded on the vessel layer. Also, the present invention is directed to a manufacturing process of the hybrid artificial blood vessel.

The artificial blood vessel of the present invention is a hybrid type wherein the biodegradable polymer-supporting layers are formed on at least one side of the inside and outside of the non-degradable artificial blood vessel layer. The biodegradable polymer-supporting layers formed on at least one side of the inside and outside of the non-degradable artificial blood vessel layer may include a drug and cause biodegradation so that the blood vessel cells can be regenerated as a patient's own blood vessel tissue.

Thus, the surface of the hybrid artificial blood vessel of the present invention in this manner substitutes the biodegradable polymer-supporting layers formed on the inside and/or outside of the vessel layer for a non-degradable artificial blood vessel layer having low cell adhesivity, resulting in improving the cell adhesivity of the artificial blood vessel. Since the cells adhered on the inside and/or outside of the biodegradable polymer-supporting layer are cultured and then regenerated as a recipient's own blood vessel tissues during the biodegradation of the polymer-supporting layer, the artificial blood vessels for tissue engineering use may be produced by inducing histogenesis on the inside and/or outside of the hybrid artificial blood vessel.

The drug is also contained in the pores of the non-degradable artificial blood vessel layer and in the biodegradable polymer-supporting layer thereon to form a biodegradable polymer-supporting layer containing the drug, whereby a hybrid artificial blood vessel containing the drug is produced. The hybrid artificial blood vessel can be varied by controlling the kind of add-on drug, the release rate of the drug, the degradation rate of biodegradable polymer-supporting layers, cell adhesivity, and the thickness of the generated tissue formed from the degradation of the supporting layer. Thus, the hybrid artificial blood vessel of the present invention can be used in a great range of application via biodegradation and local drug transmission.

In the hybrid artificial blood vessel of the present invention, materials for the biodegradable polymer-supporting layer formed on the non-degradable artificial blood vessel layer may comprise at least one polymer selected from the group consisting of synthetic polymers, such as polyglicolide, polylactide, poly(lactic-co-glicolic acid) and polycaprolactone, or natural polymers, such as chitosan, gelatin, alginic acid, hyaluronic acid and collagen. The material is preferably porous.

For the hybrid artificial blood vessel of the present invention, the non-degradable artificial blood vessel layer comprises preferably polyurethane derivatives, DacronR or drawn polytetrafluoroethylene (ePTFE). More preferably, it is ePTFE.

As described above, the hybrid artificial blood vessel of the present invention may further comprise a drug which may be stored in at least one region selected from the group consisting of the microporous space of the non-degradable artificial blood vessel layer, the biodegradable polymer-supporting layers, and the interfaces of the artificial blood vessel layer and the supporting layers. Examples of such a drug comprise at least one drug selected from the group consisting of a drug promoting tissue generation and cell growth by acting a signal on cell culture, such as vascular endothelial growth factor, fibroblast growth factor, nerve growth factor, platelet-derived growth factor and so forth; a drug acting as a signal controlling the interaction with blood or blood cell such as heparin, thrombin, and so forth; and a drug improving cell adhesion such as laminin, fibronectin, collagen and so forth.

Preferably, the biodegradable polymer-supporting layer is repetitively coated on the artificial blood vessel layer.

Furthermore, the surface of the non-degradable artificial blood vessel layer is preferably modified physically or chemically.

The above-mentioned hybrid artificial blood vessel of the present invention is obtained by dissolving biodegradable polymer in organic solvents to prepare biodegradable polymer solution A; adding porogen to the polymer solution A; dissolving the same or different biodegradable polymer with the above biodegradable polymer in organic solution to prepare biodegradable polymer solution B; incorporating the biodegradable polymer solution B into the micropore of artificial blood vessel layers; inserting tubes to the inside and outside of the artificial blood vessel layers; filling the biodegradable polymer solution A in the space between the artificial blood vessel layer and the tubes; drying the artificial blood vessel to remove the organic solvent; and incubating the artificial blood vessel in a water bath to remove the porogen.

The biodegradable polymer may contain a drug in advance.

Preferably, the porogen may comprise ammonium bicarbonate or sodium chloride.

The organic solvents for dissolving the biodegradable polymer may use any solvents conventionally available for dissolving the polymer and is not limited in this regard. Preferably, dichloromethane or dioxane can be used.

The biodegradable polymer is possible to use a biodegradable polymer bead containing a drug.

For example, a method of incorporating the biodegradable polymer solution B into the micropore of the non-degradable artificial blood vessel layer comprises blocking the alternative terminal of the non-degradable artificial blood vessel layer with tourniquets, placing the biodegradable polymer solution B with/without a drug in a syringe, and filtering the solution B into the artificial blood vessel layer so that the biodegradable polymer can be incorporated into the micropore of the artificial blood vessel wall.

To fill the biodegradable polymer solution A into the space formed between the artificial blood vessel layer and the tubes, the alternative terminals of the non-degradable artificial blood vessel layer and of the tubes inserted into the inside and outside of the artificial blood vessel layer are aligned, the aligned terminals of the tubes and the artificial blood vessel layer are enclosed with Teflon film and tape to form a configuration in which the alternative terminals of two tubes and of the blood vessel layer are blocked while having the same axis, and then the aligned configuration upends to place the blocked terminals in the bottom, followed by filling the biodegradable polymer solution A into the empty space in the inside and outside of the artificial blood vessel layer.

According to such a method, the hybrid artificial blood vessel which the biodegradable polymer-supporting layers with and/or without a drug are formed on the surfaces inside and outside the non-degradable artificial blood vessel layer and in the micropore of the blood vessel wall can be produced.

### EXAMPLES

The present invention as described above will be further exemplified by the following specific examples and experimental examples which are provided by way of illustration and not for limitation thereof.

### Example 1

### Preparation of PLGA/Chitosan/ePTFE Hybrid Artificial Blood Vessel

0.6 g of PLGA[Poly(Lactic-co-Glycolic Acid): 75:25] was added to a vial for 20 ml containing 6 ml of dioxane and dissolved for 3 hours at an ambient temperature with stirring, then 6 g of ammonium bicarbonate was added to the solution. Similarly, chitosan was added to a vial for 20 ml containing 0.2M of acetic acid solution to produce 1% of a chitosan solution.

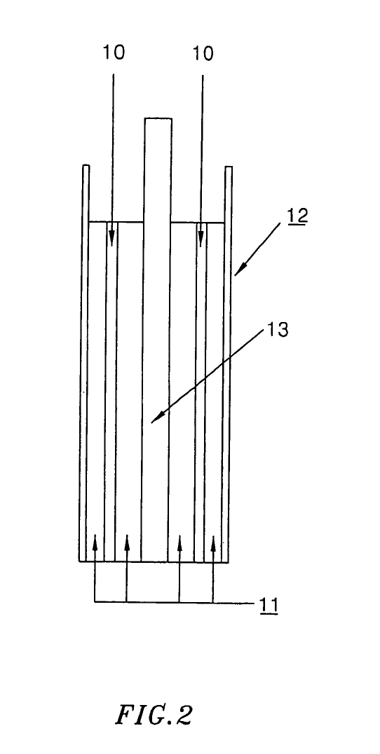

The 1% of chitosan solution was filtered through ePTFE artificial blood vessel having 6 mm of inner diameter in order to load the solution in the micropore of ePTFE wall. As shown in FIG. 2, the ePTFE artificial blood vessel containing chitosan solution was arranged with two plastic tubes having different diameters (one tube having 4 mm of outer diameter and another tube having 10 mm of inner diameter) on the same axis in order to make a mould.

The PLGA solution was added to the space between the plastic tube having 4 mm of outer diameter and the inside of ePTFE artificial blood vessel and between the outside of ePTFE artificial blood vessel and the plastic tube having 10 mm of inner diameter in order to make a mould containing the biodegradable polymer. After drying the formed mould, porogen in the mould made of the plastic tubes was removed to produce a hybrid artificial blood vessel which biodegradable supports having pore therein are formed on the inside and outside of ePTFE artificial blood vessel.

### Example 2

### Preparation of PLGA/Gelatin/ePTFE Hybrid Artificial Blood Vessel

A hybrid ePTFE artificial blood vessel was prepared as in example 1 except gelatin was substituted for chitosan.

### Example 3

### Preparation of PLGA/Hyaluronic Acid/ePTFE Hybrid Artificial Blood Vessel

A hybrid ePTFE artificial blood vessel was prepared as in example 1 except hyaluronic acid was substituted for chitosan.

### Example 4

### Preparation of a Hybrid Artificial Blood Vessel Using Sodium Chloride as a Porogen

A hybrid ePTFE artificial blood vessel was prepared as in example 1 except sodium chloride was substituted for ammonium bicarbonate.

### Example 5

### Preparation of a Hybrid Artificial Blood Vessel Using a Biodegradable Support Supplemented with Biodegradable Beads Containing a Drug

A hybrid ePTFE artificial blood vessel was prepared as in example 1 except biodegradable beads containing a drug were substituted for PLGA solution.

### Example 6

A hybrid ePTFE artificial blood vessel was prepared as in example 1 except chemically modified ePTFE was substituted for ePTFE.

### Experimental Example 1

Determination of Morphological Changes of a Hybrid ePTFE Artificial Blood Vessel

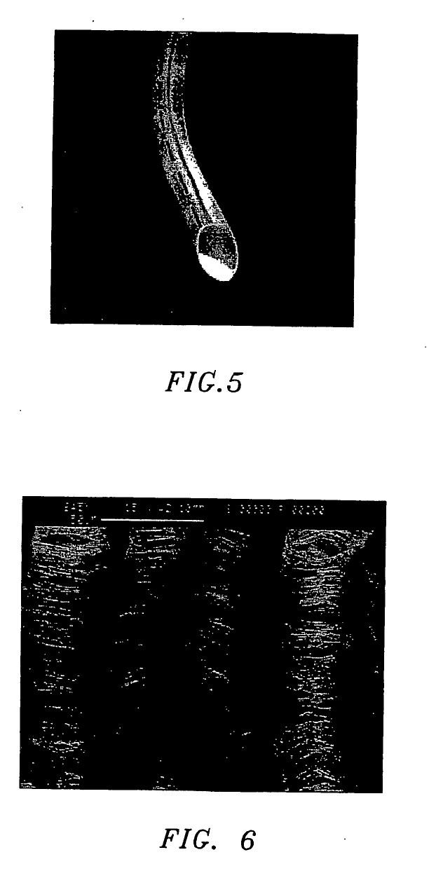

Using scanning electron microscopy, the morphological changes after a hybrid ePTFE artificial blood vessel was supplemented with biodegradable supports were determined. FIG. 5 is a scanning electron micrograph showing a section of an ePTFE artificial blood vessel before adding biodegradable polymer supports. FIG. 3 is a scanning electron micrograph showing a section of an ePTFE artificial blood vessel supplemented with biodegradable polymer supports according to example 1. As shown in FIGS. 3 and 5, it was confirmed that only ePTFE was existed before the preparation of the support, but the porous, biodegradable support was supplemented in the inside and outside of ETPFE artificial blood vessel after the preparation of the support. It was also recognized that the supports were supplemented on the outside surface of ePTFE artificial blood vessel and were porous.

### Experimental Example 2

Determination of the Ability of Biodegradation Resulting from a Hybrid ePTFE Artificial Blood Vessel

To examine the degradation phenomenon of the biodegradable support present in a hybrid artificial blood vessel, the prepared hybrid artificial vessel was dipped in a buffer solution (pH 7.0) at an ambient temperature and observed for the degradation phenomenon over 1 month. Sample which suffered from the degradation by hydration was dried in a desiccator and the weight changes of dried sample were determined every a interval. Similarly, the dried weight of another sample was determined as in the above manner except the sample was dipped in a weak acid solution. From the result, it was confirmed that the weight of the samples was gradually reduced due to the formation of the artificial blood vessel supplemented with the biodegradable supports.

### Experimental Example 3

Chemical Composition of ePTFE Artificial Blood Vessel Supplemented with Biodegradable Supports

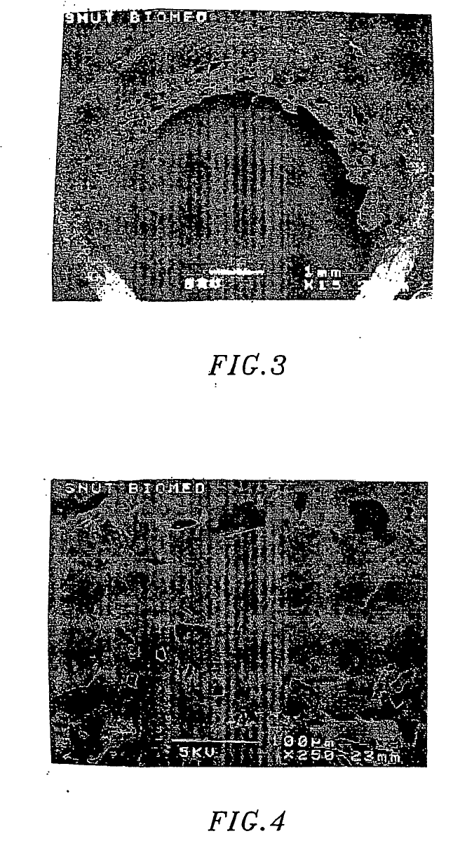

The changes of chemical composition in the surface of a hybrid ePTFE artificial blood vessel were analyzed by X-ray photoelectron microscopy after adding biodegradable supports. From the X-ray photoelectron microscopies for the surface of ePTFE artificial blood vessel shown in the FIGS. 4 and 6, it was found that the surface was comprised of fluorine and carbon only. However, no fluorine was detected from ePTFE artificial blood vessel supplemented with the supports although fluorine is a chemical component of ePTFE. It was confirmed that the ePTFE artificial blood vessel supplemented with the supports was comprised only of carbon and oxygen, which are the chemical components of PLGA. From the result, it was demonstrated that a hybrid artificial blood vessel supplemented with biodegradable supports had been formed.

### Experimental Example 4

Culturing Experiment of Fibroblast on the Surface of a Hybrid Artificial Blood Vessel

To confirm the biocompatibility of the ePTFE artificial blood vessel after modifying the surface of the vessel, fibroblast was cultured on the ePTFE artificial blood vessel before modifying the surface of the vessel and that after modifying the surface of the vessel for comparison. In the ePTFE artificial blood vessel before modifying the surface and a sample supplemented with the supports according to the method of example 1, it was shown that cell adhesivity was significantly increased. Also, on the sample before modifying the surface of the vessel and on the sample after modifying the surface of the vessel, fibroblast (1×106 cell/cm2) was directly cultured independently or fibroblast (1×106 cell/cm2) was independently cultured after adsorbing cell adhesion protein such as fibronectin. In the case of ePTFE artificial blood vessel, the inductive phenomenon of cell adhesion was observed on up to 5% of the surface area of vessel. However, in the ePTFE artificial blood vessel supplemented with biodegradable supports according to the method of example 1, it was confirmed that cell adhesivity had been significantly increased and induced over 80% of the surface area of the blood vessel.

### Experimental Example 5

Determination of Drug Transmission from a Hybrid ePTFE Artificial Blood Vessel Containing a Drug

The drug release property of a hybrid ePTFE artificial blood vessel was examined using Tritium-linked H3-Heparin. From the result, it was confirmed that the drug had been slowly released from the micropore of the ETPFE artificial blood vessel.

As described in above, the present invention provides a hybrid artificial blood vessel and a manufacturing process of the same which represents the improved patency of an artificial blood vessel due to the generation of new vascular tissues. The hybrid artificial blood vessel of the present invention allows degrading the biodegradable polymer supports with the passage of time while generating new vascular tissues.

### Industrial Applicability

Although a preferred embodiment of the present invention has been described for illustrative purposes, those skilled in the art will appreciate that various modifications, additions and substitutions are possible, without departing from the scope and spirit of the invention as disclosed in the accompanying claims.

1. A hybrid artificial blood vessel comprising a biodegradable polymer-supporting layer on at least one of an inside and an outside of a non-degradable artificial blood vessel layer, wherein a drug is stored in at least one region selected from the group consisting of the microporous space of the non-degradable artificial blood vessel layer, the biodegradable polymer-supporting layer, and the interface of the artificial blood vessel layer and the supporting layer.

2. The hybrid artificial blood vessel as claimed in claim 1, wherein the biodegradable polymer comprises at least one polymer selected from the group consisting of synthetic polymers such as polyglicolide, polylactide, poly(lactic-co-glicolic acid) and polycaprolactone, or natural polymers such as chitosan, gelatin, alginic acid, hyaluronic acid and collagen.

3. The hybrid artificial blood vessel as claimed in claim 1, wherein the non-degradable artificial blood vessel layer comprises polyurethane derivatives, DacronR or drawn polytetrafluoroethylene.

5. The hybrid artificial blood vessel as claimed in claim 1, wherein the drug comprises at least one selected from the group consisting of vascular endothelial growth factor, fibroblast growth factor, nerve growth factor, platelet-derived growth factor, heparin, thrombin, laminin, fibronectin and collagen.

6. The hybrid artificial blood vessel as claimed in claim 1, wherein the biodegradable polymer-supporting layer is porous.

7. The hybrid artificial blood vessel as claimed in claim 1, wherein the biodegradable polymer-supporting layer is repetitively coated on the artificial blood vessel layer.

8. The hybrid artificial blood vessel as claimed in claim 1, wherein the surface of the non-degradable artificial blood vessel layer is modified physically or chemically.

9. A manufacturing process of a hybrid artificial blood vessel, comprising the steps of:
dissolving biodegradable polymer in organic solvent to prepare biodegradable polymer solution A;
adding porogen to the polymer solution A;
dissolving the same or different biodegradable polymer with the above biodegradable polymer in organic solvent to prepare biodegradable polymer solution B;
incorporating the biodegradable polymer solution B into micropores of an artificial blood vessel layer;
inserting tubes to the inside and outside of the artificial blood vessel layer;
filling the biodegradable polymer solution A in a space between the artificial blood vessel layer and the tubes;
drying the artificial blood vessel layer filled with the biodegradable polymer solution A to remove the organic solvent; and
incubating the artificial blood vessel layer filled with the biodegradable polymer solution A in a water bath to remove the porogen.

10. The manufacturing process as claimed in claim 9, wherein the biodegradable polymer comprises at least one polymer selected from the group consisting of polyglicolide, polylactide, poly(lactic-co-glicolic acid), chitosan, gelatin, alginic acid and collagen.

11. The manufacturing process as claimed in claim 9, wherein the non-degradable artificial blood vessel layer comprises polyurethane derivatives, DacronR or drawn polytetrafluoroethylene.

12. The manufacturing process as claimed in claim 9, wherein the biodegradable polymer solutions A and B further comprise a drug which contains growth factors or extracellular matrices.

13. The manufacturing process as claimed in claim 12, wherein the drug comprises at least a drug selected from the group consisting of vascular endothelial growth factor, fibroblast growth factor, nerve growth factor, platelet-derived growth factor, heparin, thrombin, laminin, fibronectin and collagen.

14. The manufacturing process as claimed in claim 9, wherein the surface of the non-degradable artificial blood vessel layer is modified physically or chemically.

15. An artificial organ for tissue engineering use prepared by any one of manufacturing processes of claims 9 and 14.
